(19) United States
(12) Patent Application Publication
(10) Pub. No.: US 2023/0264046 A1
(43) Pub. Date: Aug. 24, 2023

(54) SYSTEMS AND METHODS FOR ASSESSING PATIENT-SPECIFIC EVOLUTION OF RESISTANCE TO THERAPY AND PROGRESSION OF DISEASE

(71) Applicant: H. LEE MOFFITT CANCER CENTER AND RESEARCH INSTITUTE, INC., Tampa, FL (US)

(72) Inventors: Heiko ENDERLING, New Tampa, FL (US); Solmaz SAHEBJAM, Tampa, FL (US); Daniel J. GLAZAR, Tampa, FL (US); Hsiang-Hsuan Michael YU, Tampa, FL (US)

(21) Appl. No.: 18/010,059

(22) PCT Filed: Jun. 18, 2021
(86) PCT No.: PCT/US21/38071

§ 371 Date: Dec. 13, 2022

Related U.S. Application Data
(60) Provisional application No. 63/040,579, filed on Jun. 18, 2020.

(51) Int. Cl. A61N 5/10; G16H 50/50; G16H 20/40

(52) U.S. Cl. CPC A61N 5/1038; G16H 50/50; G16H 20/40; A61N 5/1031

(57) ABSTRACT

Systems and methods for assessing patient-specific evolution of resistance to therapy and progression of disease in recurrent high-grade glioma patients are described herein. An example method includes receiving a plurality of patient-specific parameters for a patient having recurrent high-grade glioma. The patient-specific parameters include an evolution of resistance rate to a In combination therapy, a pre-treatment tumor volume, and a radiation surviving fraction. The method also includes simulating, for each of a plurality of radiation therapy protocols, a respective volumetric tumor growth trajectory for the patient. The simulation is performed using a tumor growth model based on the patient-specific parameters. The method further includes determining an optimal radiation therapy protocol based on the simulation, wherein the optimal radiation therapy prolongs progression of the recurrent high-grade glioma.

### CROSS-REFERENCE TO RELATED APPLICATIONS

This application claims the benefit of U.S. provisional patent application No. 63/040,579, filed on Jun. 18, 2020, and titled “PREDICTING GLIOMA TREATMENT RESPONSE,” the disclosure of which is expressly incorporated herein by reference in its entirety.

### BACKGROUND

Patients with recurrent high-grade glioma (HGG), such as glioblastoma, face a dismal prognosis with median overall survival rates of less than one year [1, 2]. This is likely related to the biological nature of these types of tumors which are characterized as fast growing, infiltrating, and frequently multifocal disease [3]. The diffuse nature of these tumors implies that any localized treatment, such as surgery or radiotherapy, inevitably fails to treat all (microscopic) disease and recurrences may hence occur either at the primary, or a distal location within the brain. According to NCCN (National Comprehensive Cancer Network) guidelines [4] there is no well defined standard of care for these patients and treatment options are limited. Hence, treatment strategy is often suggested on an individualized basis. These include re-resection of the tumor, systemic therapy such as bevacizumab, lomustine, or temozolomide, and palliative re-irradiation. Notably, re-irradiation in the recurrent HGG setting may be considered as a Category 2B option implying that there is NCCN consensus that this intervention is appropriate based upon lower level evidence. Recently, alternative approaches incorporating immunotherapy [5], have been tested for recurrent HGG in several clinical trials (see Laub et al. for an extensive review [6,7]) but the efficacy of this treatment could not be demonstrated. Inevitably, HGG tumors develop resistance to these systemic therapies.

### SUMMARY

An example computer-implemented method for assessing patient-specific evolution of resistance to therapy and progression of disease in recurrent high-grade glioma patients is described herein. The method includes receiving a plurality of patient-specific parameters for a patient having recurrent high-grade glioma. The patient-specific parameters include an evolution of resistance rate to a combination therapy, a pre-treatment tumor volume, and a radiation surviving fraction. The method also includes simulating, for each of a plurality of radiation therapy protocols, a respective volumetric tumor growth trajectory for the patient. The simulation is performed using a tumor growth model based on the patient-specific parameters. The method further includes determining an optimal radiation therapy protocol based on the simulation, wherein the optimal radiation therapy prolongs progression of the recurrent high-grade glioma.

In some implementations, the plurality of radiation therapy protocols include hypofractionated stereotactic radiotherapy (HFSRT) and intermittent high dose radiotherapy (iRT).

Alternatively or additionally, iRT is a high dose per fraction administered on a plurality of non-consecutive days. Optionally, a time interval between radiation therapy treatments is between about 30 and 60 days.

Alternatively or additionally, in some implementations, the optimal radiation therapy protocol is iRT, and the step of determining the optimal radiation therapy protocol includes determining at least one of a dose per fraction, a number of fractions, or a time interval between radiation therapy treatment.

Alternatively or additionally, in some implementations, the simulation is performed using the tumor growth model based on the patient-specific parameters and at least one of a tumor growth rate in an absence of therapy or an initial sensitivity to the combination therapy. Optionally, at least one of the tumor growth rate in the absence of therapy or the initial sensitivity to the combination therapy is patient-specific.

Alternatively or additionally, in some implementations, the combination therapy comprises immunotherapy and anti-angiogenic therapy.

Alternatively or additionally, in some implementations, the recurrent high-grade glioma is glioblastoma.

An example method for treating a patient with recurrent high-grade glioma is also described herein. The method includes determining an optimal radiation therapy protocol as described herein, and administering the optimal radiation therapy to the patient. For example, in some implementations, the optimal radiation therapy is intermittent high dose radiotherapy (iRT).

Additionally, the method further includes administering a combination therapy to the patient in conjunction with iRT.

Alternatively or additionally, in some implementations, the combination therapy comprises immunotherapy and anti-angiogenic therapy.

Alternatively or additionally, in some implementations, the recurrent high-grade glioma is glioblastoma.

An example system for assessing patient-specific evolution of resistance to therapy and progression of disease in recurrent high-grade glioma patients is also described herein. The system includes a processor and a memory operably coupled to the processor, where the memory having computer-executable instructions stored thereon. The processor is configured to receive a plurality of patient-specific parameters for a patient having recurrent high-grade glioma. The patient-specific parameters include an evolution of resistance rate to a combination therapy, a pre-treatment tumor volume, and a radiation surviving fraction. The processor is also configured to simulate, for each of a plurality of radiation therapy protocols, a respective volumetric tumor growth trajectory for the patient. The simulation is performed using a tumor growth model based on the patient-specific parameters. The processor is further configured to determine an optimal radiation therapy protocol based on the simulation, where the optimal radiation therapy prolongs progression of the recurrent high-grade glioma.

In some implementations, the plurality of radiation therapy protocols include hypofractionated stereotactic radiotherapy (HFSRT) and intermittent high dose radiotherapy (iRT).

Alternatively or additionally, iRT is a high dose per fraction administered on a plurality of non-consecutive days. Optionally, a time interval between radiation therapy treatments is between about 30 and 60 days.

Alternatively or additionally, in some implementations, the optimal radiation therapy protocol is iRT, and the step of determining the optimal radiation therapy protocol includes determining at least one of a dose per fraction, a number of fractions, or a time interval between radiation therapy treatment.

Alternatively or additionally, in some implementations, the simulation is performed using the tumor growth model based on the patient-specific parameters and at least one of a tumor growth rate in an absence of therapy or an initial sensitivity to the combination therapy. Optionally, at least one of the tumor growth rate in the absence of therapy or the initial sensitivity to the combination therapy is patient-specific.

Alternatively or additionally, in some implementations, the combination therapy comprises immunotherapy and anti-angiogenic therapy.

Alternatively or additionally, in some implementations, the recurrent high-grade glioma is glioblastoma.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow diagram illustrating example operations for assessing patient-specific evolution of resistance to therapy and progression of disease in recurrent high-grade glioma patients according to an implementation described herein.

FIG. 2 is an example computing device.

FIGS. 3A-3B are an overview of the data used in the examples provided below. FIG. 3A is a schematic of the NCT02313272 protocol indicating the imaging and treatment time point for this triple combination therapy trial. FIG. 3B illustrates that out of the 32 trial participants only those 16 with monitored tumor progression were included in this analysis.

FIG. 4 includes TABLE 1, Characteristics of the 16 included patients. Abbreviations: WT: wild type, Surg: surgery, TMZ: temozolomide.

FIG. 5 includes TABLE 2, Overview of the model parameters, relevant fit bounds and range of data used for fitting (patient specific, or all patients as a whole). We allowed for a wide range of growth rates with biologically reasonable limits. Bounds for ε followed previous work, and the full range of possible values was used for the surviving fraction S. A 30% volume deviation was considered for the initial tumor volume.

FIG. 6 includes TABLE 3, Overview of the predicted times to progression in days for HFSRT with bootstrap uncertainty range, and difference in time to progression for iRT and iRT+boost treatments with indicated numbers of fractions.

FIGS. 7A-7D illustrate model fit results. FIG. 7A) Grid search results to identify the optimal growth rate A for the patient population (indicated by red arrow). Results of the sum over the median, mean and maximum RMSE are shown (denoted as RMSE score). FIG. 7B) Overview of the measured vs. simulated tumor volume. FIG. 7C) Correlation analysis of the surviving fraction and the PTV gEUD. The Pearson correlation coefficient p and corresponding p-value p are given. See D for legend. FIG. 7D) Correlation analysis of the logarithm of the decay rate (log(ε)) and the surviving fraction. Abbreviations: RMSE: Root mean squared error, PTV: Planning target volume, gEUD: generalized equivalent uniform dose.

FIGS. 8A-8C illustrate evaluation of noninferiority of iRT+/−boost vs HFSRT. FIG. 8A) Kaplan-Meier plot for five treatment fractions delivered as HFSRT (red), iRT (blue) or iRT+boost (green). Shaded areas correspond to the envelope of the bootstrap estimated modelling uncertainty. The logrank test p-values is given. FIG. 8B) Decay rate parameter E for iRT responders and non-responders. FIG. 8C) Surviving fraction for iRT responders and non-responders. In FIGS. 8B) and C) the horizontal lines indicate the mean of the scores and t-test p-values are reported.

FIGS. 9A-9D illustrate Kaplan-Meier plots for treatments with increasing maximum number of iRT fractions. Shown are fitted HFSRT (red), and simulated iRT (blue) and iRT+boost (green) results. Shaded areas correspond to the envelope of the bootstrap estimated modelling uncertainty. The logrank test p-values is given. FIG. 9A) Up to seven fractions. FIG. 9B) Up to nine fractions. FIG. 9C) Up to eleven fractions. FIG. 9D) Up to thirteen fractions.

FIGS. 10A-10F illustrate grouping of patient response. FIGS. 10A-10D illustrate estimated growth trajectories of representative patients for fitted HFSRT (red), and simulated iRT (blue) and iRT+boost (green) treatments with up to 11 treatment fractions. Shaded areas correspond to the envelope of the bootstrap estimated modelling uncertainty. FIG. 10A) Group 1, FIG. 10B) Group 2, FIG. 10C) Group 3, FIG. 10D) Group 4. FIG. 10E) Analysis of the decay rate ε for the different groups. t-test p-values of the logarithm of E between groups are given. FIG. 10F) Analysis of the radiotherapy surviving fraction for the different groups. There were no significant differences.

FIG. 11 illustrates Akaike Information Criterion (AIC) was calculated as described by Portet et al. based on the residual sum of squares³³ for each of the following models: 1) γ₀=λ, fit λ per patient, 2) Fit γ₀ and λ per patient, 3) λ constant for all patients, γ₀=λ, 4) Fit γ₀ per patient, λ constant for all patients. The obtained AIC values are shown where possible given the number of data points for each patient. Model 3 (red) provided the minimal total AIC summed over all 16 patients.

FIG. 12 illustrates a local sensitivity analysis of the model parameters was performed⁶⁰ to determine how the volume tumor volume V changed in response to small perturbations in the model parameters S, ε and V₀. Mathematically, this is given by ∂V/∂p, where p=S, ε,V₀. The ranked sensitivities for each individual patient are shown. We observe that ε is the most sensitive parameters, followed by the surviving fraction S, and the pre-treatment volume V₀ for all patients but #12.

FIG. 13 illustrates an overview of model predictions for different times between fractions ranging from four to ten weeks. Reported p-values correspond to log-rank testing between HFSRT and iRT+boost treatments.

FIG. 14 includes TABLE S1, Overview of the patient-specific dosing in terms of PTV gEUD and D98%. A Lyman parameter of −10 was used for gEUD calculation. Treatments were delivered within five daily fractions for all patients. Abbreviations: PTV: Planning target volume, gEUD: generalized equivalent uniform dose, D98% near minimum dose at least received by 98% of the PTV.

FIG. 15 illustrates estimated growth trajectories of all included patients for fitted HFSRT (red), and simulated iRT (blue) and iRT+boost (green) treatments with up to 11 treatment fractions. Shaded areas correspond to the envelope of the bootstrap estimated modelling uncertainty.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

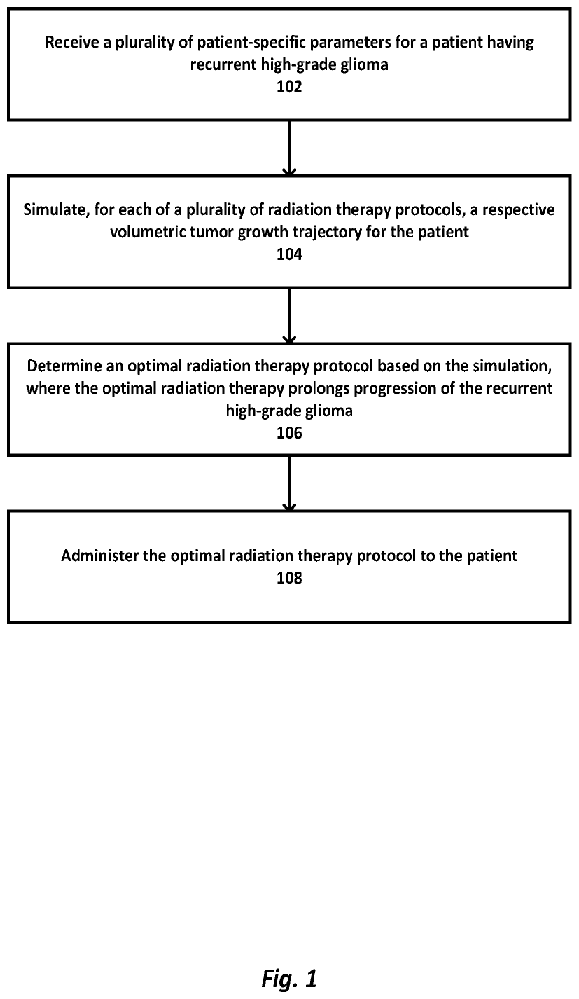

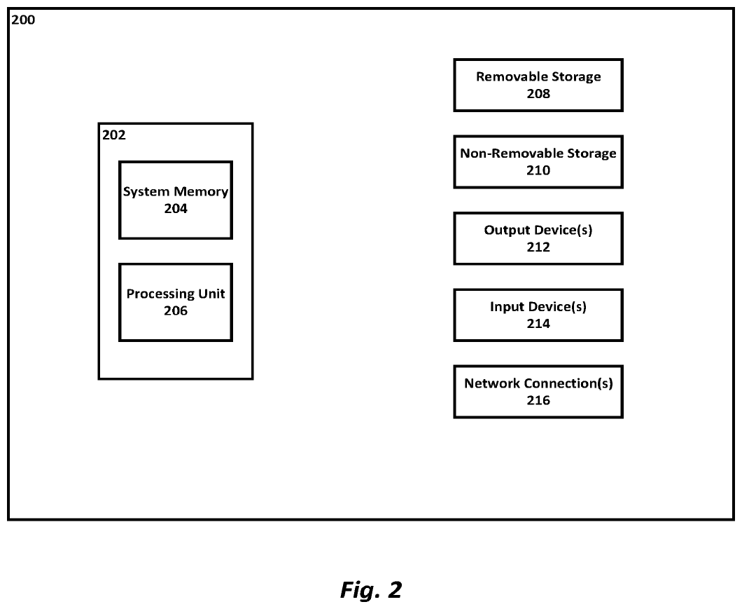

Referring now to FIG. 1, a flow chart illustrating a method for assessing patient-specific evolution of resistance to therapy and progression of disease in recurrent high-grade glioma (HGG) patients is shown. As described below, this disclosure contemplates that the method shown in FIG. 1 can be performed using a computing device such as the computing device shown in FIG. 2. Patients having recurrent high-grade glioma face poor prognosis. In fact, there are currently no curative treatment options available. As described herein, the methods can result in prolonged time to progression of disease in a recurrent high-grade glioma patient through administration of intermittent high dose radiotherapy (iRT) when used in conjunction with a combination therapy of immuno- and anti-angiogenic therapies. In particular, the methods described herein can be used to identify those patients that would benefit from iRT. Moreover, such determination can be made before or at an early time point in the patient's treatment, for example, using a simulation analysis based on a tumor growth model and using patient-specific parameters. The methods described herein therefore provide a promising personalized treatment option for recurrent high-grade glioma patients. Additionally, the high-grade glioma may be glioblastoma. It should be understood that glioblastoma is provided only as an example HGG.

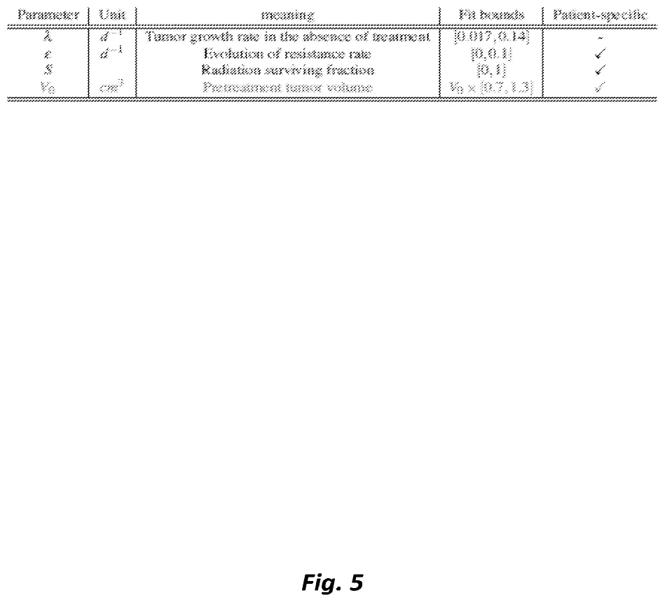

At step 102, a plurality of patient-specific parameters for a patient having recurrent high-grade glioma are received, for example, at a computing device such as the computing device of FIG. 2. As used herein, patient-specific means the parameter is specific to an individual patient. It should be understood that patient-specific is as opposed to a global, patient-uniform parameter for a plurality of patients. Patient-specific parameters can include an evolution of resistance rate (ε) to a combination therapy, a pre-treatment tumor volume (V₀), a radiation surviving fraction (S), a tumor growth rate in an absence of therapy (λ), and an initial sensitivity to the combination therapy (γ). For example, patient-specific parameters are shown in FIG. 5. In some implementations described herein, the patient-specific parameters include an evolution of resistance rate (ε) to a combination therapy, a pre-treatment tumor volume (V₀), and a radiation surviving fraction (S). In this implementation, a tumor growth rate in an absence of therapy (λ) and/or an initial sensitivity to the combination therapy (γ) are global, patient-uniform parameters. In other implementations, the patient-specific parameters include an evolution of resistance rate (ε) to a combination therapy, a pre-treatment tumor volume (V₀), a radiation surviving fraction (S), and a tumor growth rate in an absence of therapy (λ). In this implementation, an initial sensitivity to the combination therapy (γ) is a global, patient-uniform parameter. In yet other implementations, the patient-specific parameters include an evolution of resistance rate (ε) to a combination therapy, a pre-treatment tumor volume (V₀), a radiation surviving fraction (S), and an initial sensitivity to the combination therapy (γ). In this implementation, a tumor growth rate in an absence of therapy (λ) is a global, patient-uniform parameter. In yet other implementations, the patient-specific parameters include an evolution of resistance rate (ε) to a combination therapy, a pre-treatment tumor volume (V₀), a radiation surviving fraction (S), a tumor growth rate in an absence of therapy (λ), and an initial sensitivity to the combination therapy (γ).

In the examples described herein, a combination therapy is administered to the patient with recurrent high-grade glioma patient. A combination therapy is a treatment modality whereby multiple therapeutic agents are combined to treat a disease. Combination therapy is well known in the art. In some implementations, the combination therapy is administration of immunotherapy and anti-angiogenic therapy. An example immunotherapy is pembrolizumab. It should be understood that pembrolizumab is only provided as an example anti PD1 antibody. This disclosure contemplates using other immunotherapies to treat recurrent HGG including, but not limited to, other antibodies that block the PD-1 receptor . An example anti-angiogenic therapy is bevacizumab. It should be understood that bevacizumab is only provided as an example VEGF inhibitor. This disclosure contemplates using other anti-angiogenic therapies to treat recurrent HGG including, but not limited to, other agents that module angiogenesis. The methods describe herein prescribe radiotherapy in addition to the combination therapy.

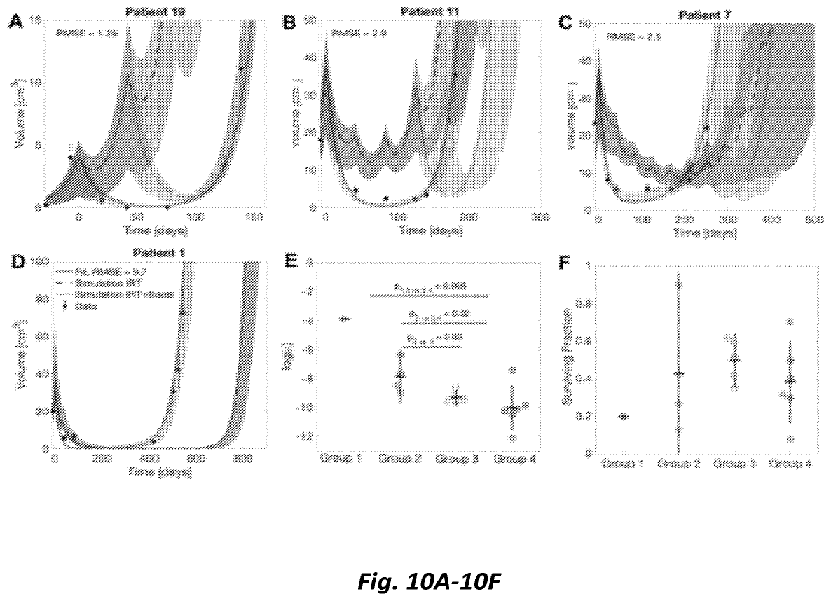

At step 104, a respective volumetric tumor growth trajectory for the patient is simulated for each of a plurality of radiation therapy protocols. This disclosure contemplates that step 104 can be performed using a computing device such as the computing device of FIG. 2. The simulation is performed using a tumor growth model based on the patient-specific parameters. An example tumor growth model is described below with regard to Equations (1)-(6), for example. FIGS. 10A-10D illustrate example simulated volumetric tumor growth trajectories for different patients according to examples described herein.

The plurality of radiation therapy protocols include hypofractionated stereotactic radiotherapy (HFSRT) and intermittent high dose radiotherapy (iRT). HFSRT involves administration of a high dose per fraction to a patient on each of ‘n’ consecutive days, where ‘n’ is an integer greater than 0. For example, a patient receiving HFSRT may be subjected to 6 Gray (Gy) of radiation on each of 5 consecutive days (e.g., Monday through Friday). It should be understood that the dose per fraction (e.g., 6 Gy) and number of doses (e.g., 5) are provided only as examples. It should be understood that the dose per fraction and/or the number of doses may have other values. HFSRT is known in the art and therefore not described in further detail herein.

In contrast to HFSRT, iRT involves administration of a high dose per fraction to a patient on each of ‘m’ non-consecutive days, where ‘m’ is an integer greater than 0. As used herein, non-consecutive means that there is a time interval between radiation therapy treatments. For example, the time interval is optionally between 30 and 60 days. This disclosure contemplates that the respective time interval between each two of the radiation treatments in the iRT protocol may the same or different. A patient receiving iRT may be subjected to 6 Gy of radiation on each of 5 non-consecutive days. A period of about 30-60 days separates each two of the radiation treatments in the iRT protocol. It should be understood that the dose per fraction (e.g., 6 Gy), number of doses (e.g., 5), and time interval (e.g., 30-60 days) are provided only as examples. It should be understood that the dose per fraction, the number of doses, and/or the time interval may have other values.

Optionally, in some implementations, iRT is accompanied by a boost. As used herein, iRT plus boost involves administration iRT plus administration of a high dose per fraction to a patient on each of ‘o’ consecutive days, where ‘o’ is an integer greater than 0, and where the boost is delivered at the time of progression. For example, the boost involves administration of 6 Gy of radiation on each of 3 consecutive days at the time of progression. It should be understood that the dose per fraction (e.g., 6 Gy) and number of doses (e.g., 3) for the boost are provided only as examples. It should be understood that the dose per fraction and/or the number of doses may have other values.

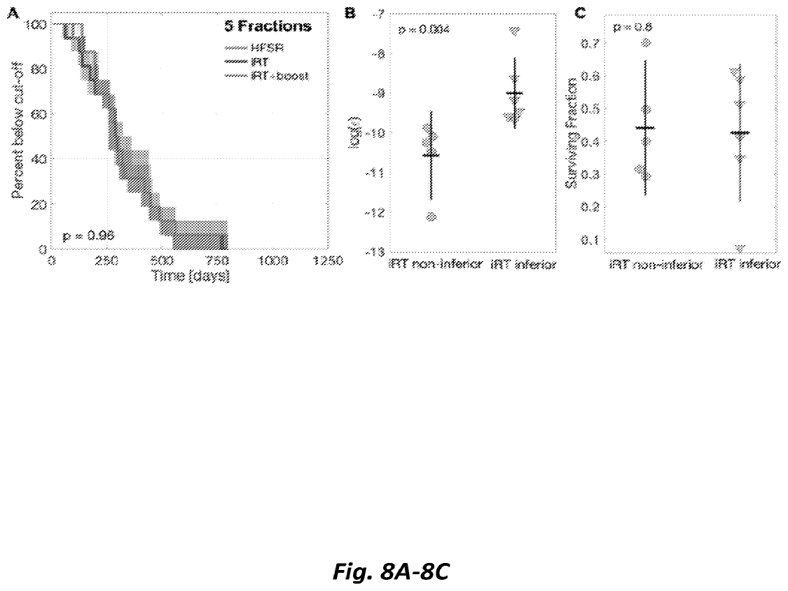

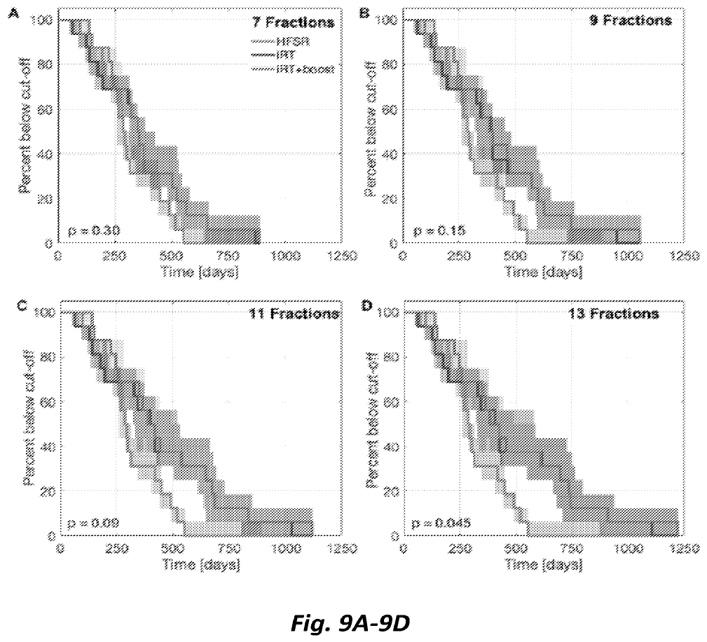

At step 106, an optimal radiation therapy protocol is determined based on the simulation. This disclosure contemplates that step 106 can be performed using a computing device such as the computing device of FIG. 2. For example, the efficacy of a radiation therapy protocol can be evaluated based on time to progression by Kaplan-Meier analysis (see FIGS. 8A and 9A-9D). Such analysis is based on changes in tumor volume (e.g. as simulated in step 104), which depend on the radiation therapy protocol. It should be understood that the optimal radiation therapy protocol prolongs progression of the recurrent high-grade glioma in the patient as compared to the non-optimal radiation therapy protocols. The methods described herein facilitate a comparison of times to progression for different radiation therapy protocols (e.g., HFSRT, iRT (with or without boost), etc.). The optimal radiation therapy protocol for a patient (e.g., personalized radiation therapy) can be determined before administering therapy (or at an early evaluation time point).

Optionally, in some implementations, the optimal radiation therapy protocol is iRT. In this case, the step of determining the optimal radiation therapy protocol optionally further includes determining at least one of a dose per fraction, a number of fractions, or a time interval between radiation therapy treatment. In this way, personalized radiation therapy can be prescribed and administered to the patient. As described in the examples herein, the dose per fraction is 6 Gy, the number of fractions is 5, and the time interval is 30-60 days. This disclosure contemplates that the step of determining may include a determination of an individualized treatment for the subject. For examples, various number of fractions for iRT (e.g., 5, 7, 9, 11, and 13 fractions as shown in FIGS. 8A and 9A-9D) are evaluated. Additionally, the methods described herein can be used to personalize the dose per fraction. Optionally, the methods described herein can be used to select a patient who will respond to iRT plus boost.

Optionally, at step 108, the optimal radiation therapy protocol is administered to the patient. Thus, the patient is treated with the optimal radiation therapy protocol in addition to the combination therapy, which includes immuno- and anti-angiogenic therapies. In other words, the patient's recurrent HGG is treated radiotherapy, immunotherapy, and anti-angiogenic therapy in combination.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Recurrent grade glioma patients face a poor prognosis for which no curative treatment option currently exists. In contrast to prescribing high dose hypofractionated stereotactic radiotherapy (HFSRT, 6 Gy×5 in daily fractions) with debulking intent, we suggest a personalized treatment strategy to improve tumor control by delivering intermittent high dose treatment (iRT, 6 Gy×1 every six weeks). We performed a simulation analysis to compare HFSRT, iRT and iRT plus boost 6 Gy×3 in daily fractions at time of progression) based on a mathematical model of tumor growth, radiation response and patient-specific evolution of resistance to additional treatments (pembrolizumab and bevacizumab). Model parameters were fitted from tumor growth curves of 16 patients enrolled in the phase 1 NCT02313272 trial that combined HFSRT with bevacizumab and pembrolizumab. Then, iRT +/−boost treatments were simulated and compared to HFSRT based on time to tumor regrowth. The modelling results demonstrated that iRT+boost(−boost) treatment was equal or superior to HFSRT in 15(11) out of 16 cases and that patients that remained responsive to pembrolizumab and bevacizumab would benefit most from iRT. Time to progression could be prolonged through the application of additional, intermittently delivered fractions. iRT hence provides a promising treatment option for recurrent high grade glioma patients.

### Introduction

Patients with recurrent high-grade glioma (HGG), such as glioblastoma, face a dismal prognosis with median overall survival rates of less than one year [1, 2]. This is likely related to the biological nature of these types of tumors which are characterized as fast growing, infiltrating, and frequently multifocal disease [3]. The diffuse nature of these tumors implies that any localized treatment, such as surgery or radiotherapy, inevitably fails to treat all (microscopic) disease and recurrences may hence occur either at the primary, or a distal location within the brain. According to NCCN (National Comprehensive Cancer Network) guidelines [4] there is no well defined standard of care for these patients and treatment options are limited. Hence, treatment strategy is often suggested on an individualized basis. These include re-resection of the tumor, systemic therapy such as bevacizumab, lomustine, or temozolomide, and palliative re-irradiation. Notably, re-irradiation in the recurrent HGG setting may be considered as a Category 2B option implying that there is NCCN consensus that this intervention is appropriate based upon lower level evidence. Recently, alternative approaches incorporating immunotherapy [5], have been tested for recurrent HGG in several clinical trials (see Laub et al. for an extensive review [6,7]) but the efficacy of this treatment could not be demonstrated. Inevitably, HGG tumors develop resistance to these systemic therapies.

A number of modeling approaches have investigated the responses of (low grade) glioma and oligodendroglioma in clinical [8-12] and preclinical [13] settings. Pérez-García et al. specifically investigated the implications of changing the radiotherapy treatment fractionation to highly protracted delivery for treatment of low grade gliomas [11] and oligodendrogliomas [9]. These analyses provided very promising, yet purely theoretical, results predicting a potential survival benefit in case of intermittent radiotherapy delivery. Intermittent RT is suggested to delay the emergence of treatment-resistant and more aggressive clones. Moreover, intermittent delivery may widen the therapeutic window between normal tissue complication and tumor control due to a superior repair capacity of normal tissue. This could enable dose escalation without necessarily increasing toxicity. There also exists increasing evidence for synergistic combination of radiotherapy with immune checkpoint inhibitors [14]. Although the underlying mechanisms of action remain subject to investigation, the immunogenic effect of radiation has been demonstrated in numerous(pre) clinical studies [15]. It is suggested that radiation induced damage triggers innate immune receptors leading to enhanced antigen presentation and immune cell activation [16]. As such, intermittently delivered radiotherapy could provide repeated immune system stimuli and could potentially be further enhanced by combination with immune checkpoint inhibitors [14]. In summary, there exists a strong motivation to further investigate protracted fractionation based on patient data and within recurrent HGG.

In our recent phase 1 clinical trial (NCT02313272, May 12, 2014) recurrent HGG patients were treated with a combination of hypofractionated stereotactic radiotherapy (HFSRT; 6 Gy×5 fractions), bevacizumab (antibody against vascular endothelial growth factor (VEGF)) and pembrolizumab (anti PD1 antibody) [8]. This study demonstrated safety in terms of adversarial side effects for this particular protocol. Although efficacy was not the primary endpoint, the response results were promising; yet median time to progression remained below one year [8]. In this trial, HFSRT was given in doses with maximum log cell kill intent over one week (consistent with current practice and trials for recurrent HGG re-irradiation studies [9]). While this dose fractionation strategy can be effective in eradicating cancer cells, it may select for radiation-resistant subclones by preferential killing of radio-sensitive subclones through an ecological-evolutionary process called competitive release [10-12]. The current protocol of maximum tolerable dose did not account for such evolutionary dynamics, and every patient inevitably developed resistance. Furthermore, HFSRT given upfront prevented the possibility to re-irradiate any additional (local or distant) recurrences, and provided only a single immune stimulus for anti PD1 treatment. Glazar et al. developed a mathematical model describing the dynamic growth response of HGG to a combination of bevacizumab and pembrolizumab aiming to predict the onset of treatment resistance [12]. Yet their approach did not account for the effects of radiotherapy and how adaptation to the fractionation schedule may improve patient outcome.

Here, we investigate through mathematical modelling an alternative, intermittent approach for radiotherapy treatment schedules. This approach is motivated by the assumption that for this group of patients tumor management will prolong time to progression compared to (failed) tumor eradication. The rational of evolutionary principles-guided intermittent treatment approach is to maintain a treatment-sensitive population that competes for resources with resistant cells and thus slows the expansion of a resistant clone, thereby prolonging time to progression [13, 14]. Moreover, when delivering radiotherapy using an intermittent schedule, treatment dosing and irradiation volume can be adapted based on observed responses.

In this example we describe a mathematical model, using only three patient specific parameters, that is suitable to fit clinically observed longitudinal, volumetric tumor growth in patients enrolled in the NCT02313272 trial. We use this model to simulate alternative, intermittent treatment schedules to determine whether these provide superior and personalizable alternatives to the current HFSRT for protocol for recurrent HGG.

### Materials and Methods

### Patient Cohort

Patients with recurrent HGG included in this modelling study (n=16) were treated at the Moffitt Cancer Center, FL between August 2015 and March 2018 as part of a phase I clinical trial (NCT02313272, May 12, 2014) [8]. All patients provided written consent and the treatment protocol was approved by the institutional review board (IRB study #: Pro00014674 and #00000971). Following optional surgical resection, all patients received HFSRT (protocol of Gy×5 delivered as five consecutive, daily fractions).

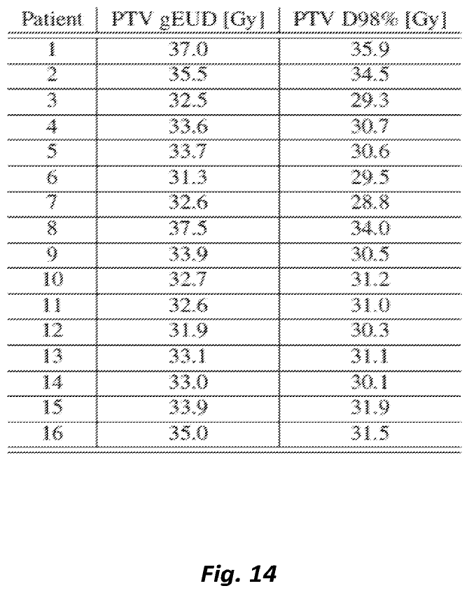

Here, treatment was prescribed as 30-35 Gy to the planning target volume (PTV) with a simultaneously integrated boost to the gross tumor volume (GTV) of D_{95%}=30-40 Gy. All treatment plans were calculated in iPlan (Version 1.1 Brainlab, Munich, Germany) and were delivered as intensity modulated radiotherapy treatments using volumetric modulated arc therapy with image guidance. Planned doses were summarized in terms of generalized equivalent uniform dose (gEUD) [15, 16] and near minimum dose D_{98%}, delivered to the PTV. gEUD ranged between 31.3 and 37.0 Gy, whereas the corresponding near minimum dose D_{98%} was between 28.5 and 35.9 Gy (see FIG. 14, TABLE S1 for specific total dose per patient). gEUD calculations were performed in Matlab (version 2020a) using an exponent (Lyman parameter) of −10 as suggested previously [17]. The gEUD accounts for dose inhomogeneity, whereas the PTV captures geometric delivery uncertainties across the gross tumor volume, providing the basis for volumetric response evaluation.

In addition to HFSRT, all patients received the VEGF inhibitor bevacizumab (10 mg/kg, intravenously delivered every two weeks) and the anti PD1 antibody pembrolizumab (100 mg or 200 mg, intravenously applied every three weeks). Both, bevacizumab and pembrolizumab were given until time of progression (scored by a 20% increase in tumor volume above the nadir as per RANO criteria [18]) or toxicity. Tumor volume was assessed pre-treatment and approximately every six weeks (median 42 days, standard deviation 38 days) using T1-weighted, contrast enhanced magnetic resonance imaging (3T MRI, 1.5 mm slice thickness). The region of hyper intensity on post-contrast T1-weighted MR images (T1 post) was contoured by a neuro-radiation oncologist as the GTV. Where required, additional MRI sequences such as T2-weighted and/or FLAIR imaging were used to accurately assess this GTV, especially when there was significant tumor associated edema. A 5 mm expansion was made from the GTV to create the PTV.

Response was assessed every 6 weeks per Response Assessment in Neuro-Oncology (RANO [27]) criteria. Radiographic progression is defined as 25% or greater increase in the sum of the products of perpendicular diameters of the enhancing lesion in T1 post, when compared with baseline or smallest tumor measurement (nadir). Additionally, progression may be observed by a significant increase in T2/FLAIR non-enhancing lesion on stable or increasing doses of corticosteroids compared with nadir. Here we evaluate tumor volumes in T1 post MRI measurements that recently demonstrated correlation with response [12].

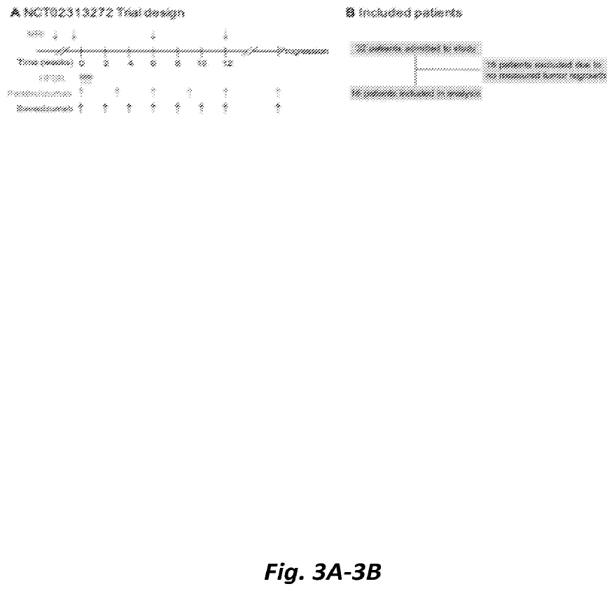

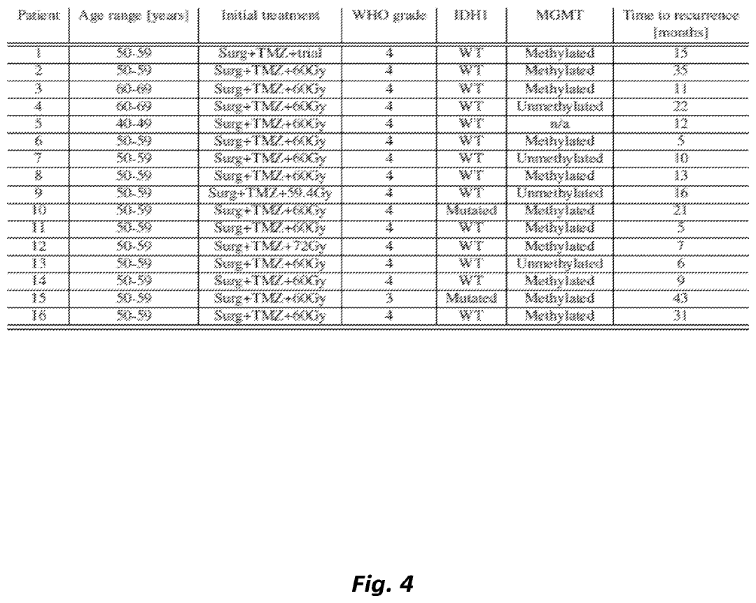

A subset of 16 trial patients (both bevacizumab naive and pretreated) with tumor measurements beyond the time of progression (i.e. tumor regrowth) was used in this study. Patients excluded from this analysis either left the trial due to reasons other than tumor progression, or their tumor regrowth was not quantified. For the selected group, four to ten (median six) post treatment data points were acquired. FIGS. 3A-3B outlines the trial design (FIG. 3A) and the patient subset included in this analysis (FIG. 3B). The patient characteristics are summarized in FIG. 4, TABLE 1. Patients are shown with arbitrary identifies.

### Mathematical Model

The aim of this study was to provide a simple mathematical framework to (i) fit the observed tumor growth response data to HFSRT given in five daily fractions, and, based on this description, to (ii) simulate intermittent radiation treatment (iRT) schedules. The presented model captures only the key mechanisms of treatment response to limit the mathematical complexity of the model to be able to obtain high confidence fit parameters estimates. We extended a mathematical tumor-growth inhibition model reported by Glazar et al. to account for the contribution of HFSRT to tumor volume reduction [19]. Tumor volume growth was described as exponential growth at rate λ[day⁻¹], hence neglecting potential plateauing effects due to limited carrying capacity of a tumor [20] within this time frame. Upon treatment initiation, the effect and onset of resistance to bevacizumab and pembrolizumab treatment was modelled as previously described [19] by exponential tumor volume reduction with rate γ[day⁻¹]:

$$\frac{{dV}_{l}}{dt} = λ {V}_{l} - γ ( l ) {V}_{l} \qquad ( 1 )$$

Here, Vₗ(t) is the viable tumor volume at time t, and γ(t) denotes the volume decay due to bevacizumab and pembrolizumab treatment. As treatment resistance builds up, this decay rate exponentially decreases at a characteristic rate ε:

$$\frac{d γ}{d t} = - ε γ \qquad ( 2 )$$

In summary this leads to the following analytic solution to this system of ordinary differential equations (ODEs):

$${V}_{l} ( t ) = {V}_{0 , l} \cdot \text{?} \\ \text{?} \text{indicates text missing or illegible when filed} \qquad ( 3 )$$

with initial conditions V_{0,l}, and γ₀ at time t₀.

To model radiotherapy effects, at each treatment fraction delivery (t_{RT}), a proportion of (1−S) of the viable tumor is transferred to a dying compartment V_{d}. The surviving fraction S is here used as a model parameter in itself, rather than as a function of radiation dose and patient specific radiation sensitivity, as described by the linear-quadratic model [21].

Vₗ(t_{RT}⁺)=S·Vₗ(t_{RT}⁻)

V_{d}(t_{RT}⁺)=V_{d}(t_{RT}⁻)+(1−S)Vₗ(t_{RT}⁻) (4)

Here, t_{RT}⁻ denotes time immediately before delivery of a radiation fraction, t_{RT}⁺ the time immediately after treatment delivery. By restricting ourselves to the same fraction size as the HFSRT treatment, the presented model provides a worst case estimate of no explicit consideration of radiation-induced immune stimulation.

We model radiation induced cell death as mitotic catastrophe [22], which is a proliferation-dependent process. Hence, we describe the volume change as an exponential reduction of V_{d}(t). Whereas others included separate parameters for the relevant tumor shrinkage rate [8, 13, 31], we assume a reduction of V_{d}(t) at rate λ identical to the growth rate to restrict the number of free parameters. This assumption is motivated on the possibility of cell death upon the attempt of cell division [32].

$$\frac{{dV}_{d}}{dt} = - λ \to {V}_{d} ( t ) = {V}_{0 , d} \text{?} \\ \text{?} \text{indicates text missing or illegible when filed} \qquad ( 5 )$$

Hence, the total, observed tumor volume V(t) comprises a proliferating (Vₗ(t)) and dying (V_{d}(t)) population.

V(t)=Vₗ(t)+V_{d}(t) (6)

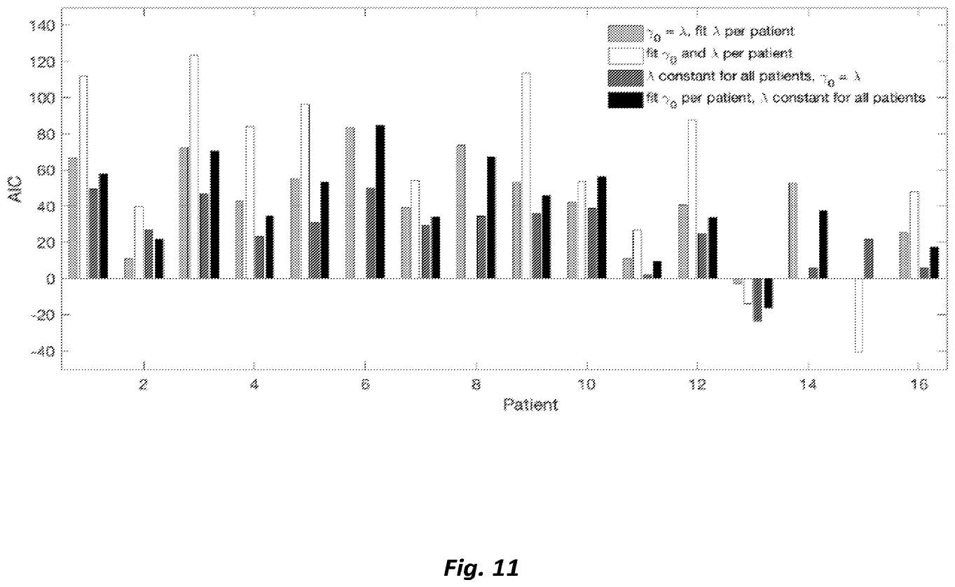

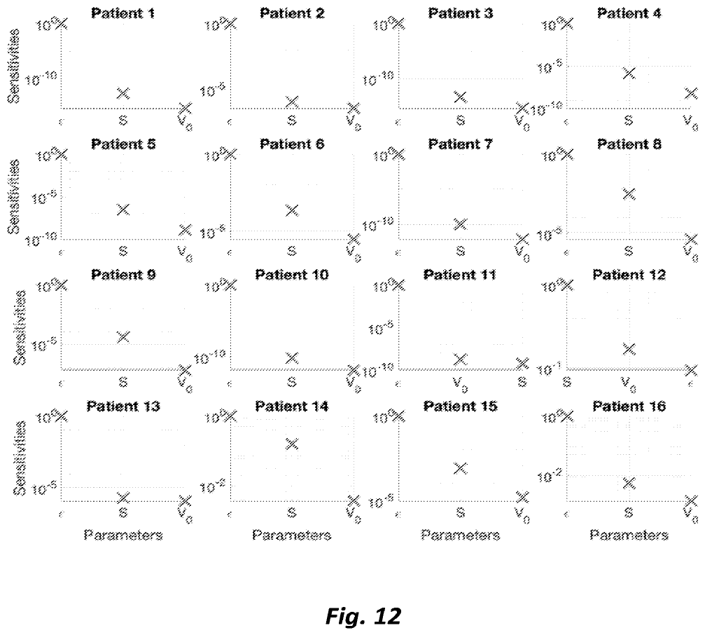

In total, this model comprises five parameters (V₀, λ, γ₀, ε, S, see also FIG. 5, Table 2). Following previous work [12], we evaluated the possibility of reducing the number of patient-specific parameters by considering global, patient-uniform parameters as well as functional dependencies between parameters. Using the Akaike Information Criterion (AIC) [33] we identified the minimized AIC with a global, patient-uniform growth rate λ and γ₀=λ (FIG. 11). Sensitivity analysis of the patient-specific parameters (S, ε, V₀) demonstrated the rate at which patients develop resistance to combination therapy with bevacizumab and pembrolizumab, ε, to be the most sensitive model parameter, and the initial tumor volume, V₀ to be the least sensitive (FIG. 12).

### Parameter Fitting and Uncertainty Estimation

All calculations and modelling were performed in MATLAB version 2020a. Agreement between clinically measured and simulated data was assessed by root mean squared error (RMSE) calculated over all data points including pre-treatment and four to ten (median six) post-RT measurements. Since more than one pre-treatment volume measurements were only available for a small subset of patients (7/16), a grid search was performed to identify the most suitable growth rate for the patient population as a whole. Doubling times ranging from five to 40 days (λ=[0.017, 0.14] day⁻¹) were used for model fitting under the constraint of a global, patient-uniform growth rate. For each growth rate, the sum of mean, median, minimum and maximum taken over all RMSEs obtained over the full data range for all patients was compared to identify the optimal growth rate. All further results are based on this optimal growth rate.

Parameters were fitted to all data for each patient by minimizing the sum of relative squared differences between simulated and measured tumor volume. We used the MATLAB function fmincon with a sqp solver, a step tolerance of 10⁻⁹, and an optimality tolerance of 10⁻⁹. All fits converged based on these cut-off criteria.

Parameter variation due to contouring uncertainty was estimated by bootstrapping. Each data point was shifted by multiplication with a random number drawn from a normal distribution of mean one and standard deviation 0.2 (hence assuming up to 20% uncertainty) before repeating the fit of ε and S. We assumed a maximum contouring uncertainty of 20% due to inter observer variation, and potential microscopic infiltration of tumor cells which was not accounted for in the original GTV definition. This was a conservative estimate based on inter observer variation reported to range between 12-55% (mean 27%) [35]. For data points below 2 cm³ a 0.5 cm³ uncertainty was used, independent of the recorded tumor volume to account for a minimal contouring uncertainty. Any potential negative data points were assigned to zero volume. Fifty bootstraps were calculated, and envelopes of the obtained results are shown as uncertainty bands. For all model parameters data is given as results of the undisturbed data with standard deviations over the bootstrap results. Correlation between model parameters, and between a patient's gEUD and estimated surviving fraction S were evaluated with MATLAB's corrcoef function.

### Comparison of Alternative Treatment Schedules

We first investigate the noninferiority of intermittent RT vs. HFSRT: Based on all estimated parameters the volumetric growth trajectory for each patient is simulated for five treatment fractions of the same dose per fraction as delivered by HFSRT but given intermittently every six weeks. The relevant patient-specific dose per fraction in terms of gEUD and D98% are listed in FIG. 14, Table S1. The interval of six weeks (42 days) corresponded to the imaging interval performed during the NCT02313272 trial and was a compromise between imaging cost and longitudinal coverage of growth observation. Previous work in low grade glioma recommended an optimal treatment interval between 30-60 days depending on the specific tumor growth rate [11]. Results were also analyzed for simulations of intermittent RT intervals of 4,8, and 10 weeks (28, 56, and 70 days). To account for treatment resistant tumors that may outgrow their pretreatment size within the six-week inter-fraction window, we also model iRT plus a three fraction boost at time of progressions (delivered in three consecutive days). As neither RANO or RECIST [36] were developed for intermittent therapy that does not aim to debulk the tumor and minimize the nadir, we here define “progression” as volume at the six-weekly assessment points exceeding the minimum measured tumor volume by more than 20%. Differences in the obtained fit parameters between patients where iRT (with or without boost) was inferior to HFSRT and those where it was equal or superior are compared by Wilcoxon rank test using MATLAB's rank sum function.

Based on the assumption of a superior repair capacity of healthy relative to tumor tissue [23], the intermittent treatment delivery holds the potential for reduced normal tissue toxicity at the same number of treatment fractions. In a second step, we hence investigate the potential gain in time to progression by increasing the number of intermittently delivered treatment fractions. We perform simulations for up to 13 treatment fractions.

The efficacy of any treatment schedule is evaluated based on time to progression by a Kaplan-Meier-analysis. Depending on the fractionation scheme the change in tumor volume varies significantly and a metric assessing both volume maintenance, and tumor eradication treatments is key. We hence score the time to reach the last recorded tumor volume (cut-off volume), assuming that this provided an estimate of the patient's maximum tolerated tumor burden. If a patient left the trial due to other reasons than increasing tumor burden, the time to reach a simulated 20% increase above the initial volume was scored. Kaplan-Meier plots were generated using the MatSurv package [24] and log-rank p-values were calculated between HFSRT and iRT+boost treatments.

### Results

### Model Fit to Data

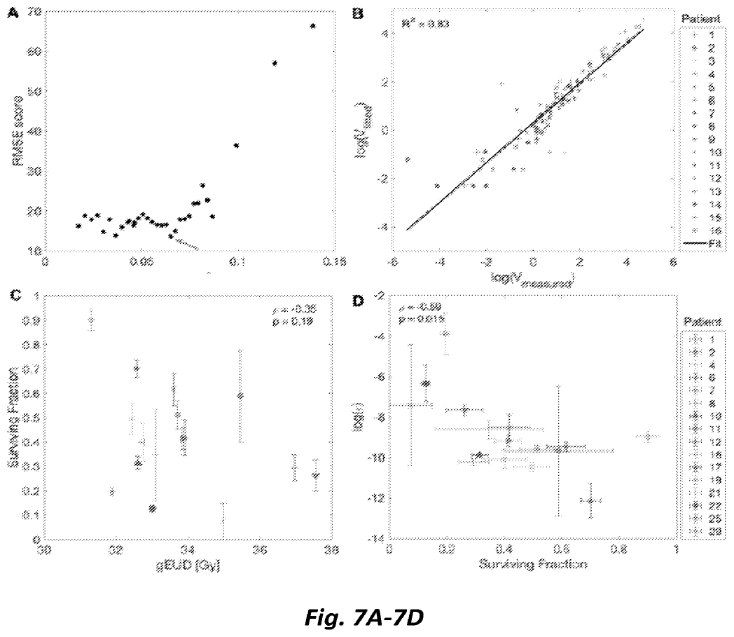

The optimal growth rate was estimated by grid search to be λ=0.065 day⁻¹, corresponding to a doubling time of 10 days (FIG. 7A). For the data fits of all 16 patients with a fixed growth rate of 0.07 d⁻¹ we obtained a median (minimum, maximum) RMSE of 1.9 (0.2,9.7). Considering the overall correlation of fitted and measured tumor volume (FIG. 7B) there was good agreement in terms of coefficients of determination (R²=0.83) which we calculated here over the logarithms of the data points to prevent an over-representation of large values in this analysis. Model fits to individual patient data are shown in FIG. 14, Table S1.

The obtained patient specific fit parameters, reported as median and full range, spanned a fairly large interval reflecting the biological heterogeneity and prescription dose variations: S=0.41 (0.07,0.90), ε=0.9 (0.05,202) 10₋₄. Interestingly, there was no correlation (p>0.05) between estimated surviving fraction and planned dose in terms of gEUD (FIG. 7C) or D98% (not shown) in the PTV, possibly reflecting the radio sensitivity heterogeneity of the tumors. FIG. 7D shows that there was a significant negative correlation (p=0.02) between S and log(ε) indicating that more radio resistant tumors, which are characterized by a high surviving fraction S, had a smaller rate of resistance development than non-RT treatments ε.

Demonstrating Non-Inferiority of iRT+Boost

We observed no significant difference between HFSRT and iRT (p=0.83), or HFSRT and iRT+boost (p=0.83) for five treatment fractions (FIG. 8A). Given the small number of patients, it is, however, also important to evaluate the individually observed differences at the patient level. In 11/16 patients, iRT was equal to HFSRT, whereas time to volume cut-off was smaller for five patients. These patients (#6,8,12,14,15) were characterized by a significantly faster decay of the non-RT treatment effects (ε_{iRT<HFSRT}=4.7 (1.3,202) 10₋₄, ε_{iRT≥HFSRT}=0.6 (0.1, 5.9) 10⁻⁴, p-value between log(ε)=0.006) leading to faster regrowth (FIG. 8B) leading to fast regrowth. There was no significant difference in the obtained radio sensitivity (indicated by S) of the two groups (S_{IRT<HFSRT}=0.26 (0.13,0.90), S_{IRT≥HFSRT}=0.41 (0.07,0.70), p=0.44) (FIG. 8C).

For four of these five patient time to progression could be prolonged by delivering a three-fraction boost once regrowth occurred. As such, Kaplan-Meier analysis (FIG. 8A) shows that for 15 out of the 16 cases, iRT+boost was noninferior to the trial's HFSRT treatment schedule. The single patient for whom iRT+boost was inferior to HFSRT treatment displayed the largest resistance evolution rate εₚₐₜᵢ₁₉=202±206·10⁻⁴ day⁻¹.

### Modelling of Extended Intermittent Treatments

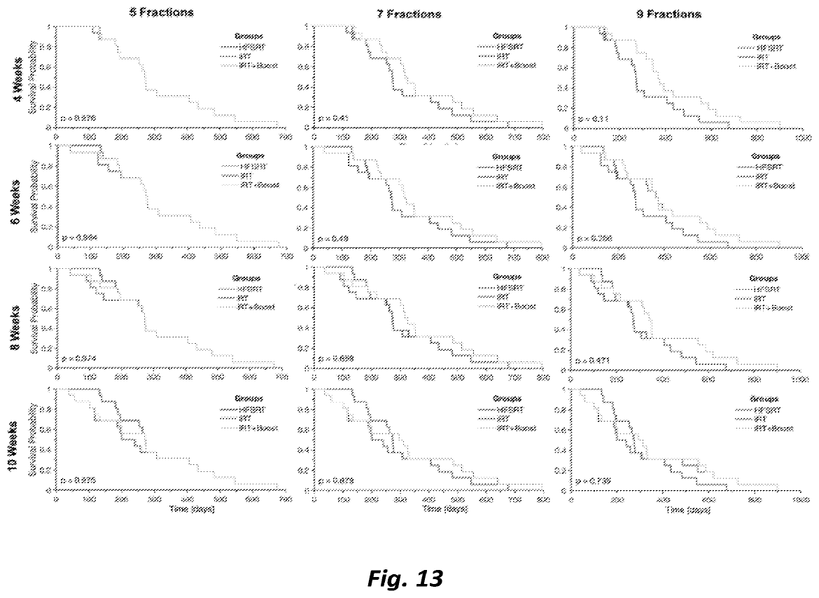

Normal and tumor tissue radio sensitivity may vary strongly between individual patients. The opportunity to either continue or interrupt treatment at each of the 6-weekly evaluation time-points holds great potential for treatment personalization. Here, the total number of delivered fractions can be adjusted leading to personalized dose escalation given the absence of acute normal tissue toxicity. In the intermittent setting, normal tissue may be capable to compensate for radiation-induced damage more effectively than the tumor, which motivates an escalation of the total delivered dose in the iRT setting. We hence simulated alternative treatments allowing up to 13 treatment fractions. As the number of fractions increases, the time to reach the cut-off volume is continuously prolonged provided the subject's tumor shows no regrowth, which in turn increases separation of the Kaplan-Meier curves as shown for up to seven (FIG. 9A), nine (FIG. 9B), eleven (FIG. 9C), or thirteen (FIG. 9D) treatment fractions. However, given the small cohort size, differences between HFSFRT and iRT+boost for up to eleven intermittent treatment fractions were not significant as assessed by logrank testing (p>=0.05). Only a subset of five patients (#1, 3, 7, 10, 11) would benefit further from >11 treatment fractions (FIG. 9D) leading to a further separation of the Kaplan-Meier plots for observation time above 550 days and significantly better iRT+boost treatment compared to HFSRT (p=0.045). Changing the timing between intermittent radiation fractions did not demonstrate a significant difference in results. However, prolonging inter-fraction times beyond six weeks may diminish potential benefits of iRT as the tumor regrowth could lead to progression before subsequent radiation (FIG. 13).

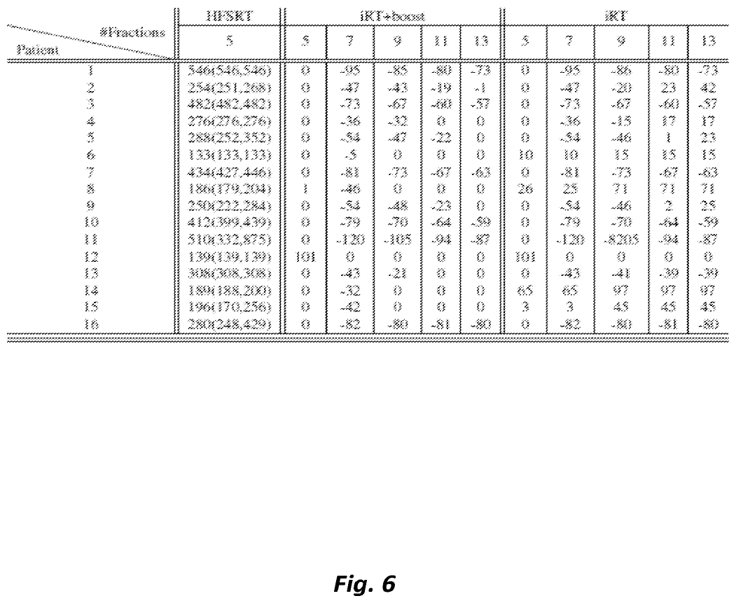

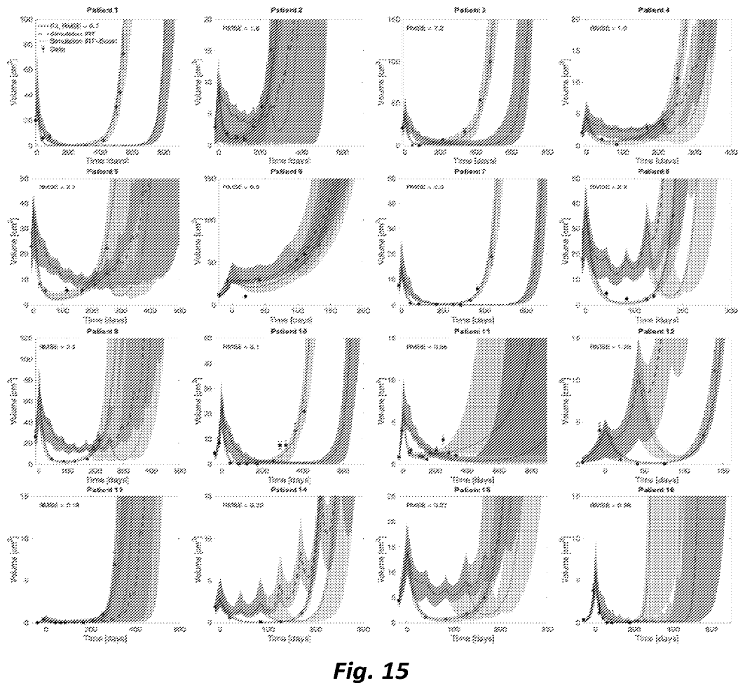

By comparing the individual patient's response between protocols (see FIG. 6, Table 3) we identified four subgroups of patients with respect to their response to the different protocols: 1) HFSRT is best (#12), 2) iRT is inferior to HFSRT, but iRT+Boost compensates this difference (#6, 8, 14, 15), 3) iRT+boost further prolongs time to cut-off volume (#2, 4, 5, 9, 13), 4) iRT is best (#1, 3, 7, 10, 11, 16). FIGS. 10A-10D shows individual growth trajectories for representative examples for each of these groups for a scenario of up to eleven iRT fractions to clearly visualize the differences in growth response. The full set of growth trajectories is given in the supplementary material (FIG. 15). The relevant evaluation of the model parameters for these sub-groups is shown in FIGS. 10E and 10F. While there was no significant difference between the radiotherapy surviving fraction, the groups differed in their fit results for parameter E with small decay rates corresponding to a benefit from iRT treatment.

### Discussion

Improving treatment responses and outcomes of recurrent high grade glioma patients is an unmet clinical need. Here we developed, analyzed, and calibrated a mathematical model to simulate and compare HFSRT with 6Gy×5 fractions delivered in one week to intermittent radiation of 6 Gy in 5 or more fractions delivered intermittently at 6 week intervals with concurrent bevacizumab and pembrolizumab. Model simulation results suggest that an intermittent treatment plus boost should be equal to or superior to the five daily fraction HFSRT protocol for all but one of the evaluated patients. We found that the parameter characterizing the rate of developing resistance to bevacizumab and pembrolizumab treatment was an indicator of the efficacy of iRT. Patients whose tumor developed resistance slowly, would benefit most from iRT compared to HFSRT. Based on our data it was not possible to identify these patients from pre-treatment or early evaluation time points. This disclosure contemplates that other biomarkers, e.g. genetic data or imaging radiomics, could be predictive of this mechanism and hence allow for stratification of patient according to the best suitable fractionation scheme. In the absence of such predictive markers, it would hence be essential to continuously monitor the tumor's response as suggested here. We showed that iRT could possibly extend the time to tumor progression by allowing for personalization of later treatment fractions based on the observed response to the previous fraction. This could be done, as suggested here, through the option of a boost in case of progression, or by adapting the dose per fraction regimen at a personalized level as previously suggested for other tumor locations [14, 20]. Fraction size variation was not addressed in this model due to the limited amount of data available. Given a more in-depth knowledge of the specific radio sensitivity parameters per tumor, for example based on genetic information, further analyses could integrate a tumor-specific radio sensitivity index [25, 26]. We intended to restrict this analysis to a purely data-driven estimation of response with few patient specific fit parameters. Genetic or molecular data was not available and hence could not be accounted for. Despite these simplifications, an interesting observation was that more radio resistant tumors generally displayed a smaller rate of resistance development than non-RT treatments leading to a significant correlation of S and ε. Since RT and immunotherapy were delivered simultaneously in the NCT02313272 trial, it cannot be determined if this is a biological result or due to mathematical parameter non-identifiability. It is generally agreed that the extent of the RT-induced immune stimulus is dose dependent, and hence proportional to the number of cells killed [14]. It could be speculated that a smaller immune stimulus resulted in a delay of the onset of treatment resistance however further biological, clinical, and mathematical analyses are needed in order to draw definite conclusions.

The proposed mathematical model, despite using only three patient-specific parameters, provided an acceptable fit to the data. Information criteria analysis showed that allowing for all 5 parameters to be patient specific may lead to better fits to the clinical data, but at the cost of overfitting of the interdependent model parameters given the sparse patient data (median six on-treatment data points per patient). Adding additional biological complexity, such as radiation-induced immune stimulation, albeit of high clinical relevance would even further limit the identifiability of parameters. By choosing a deliberately simple mathematical description, the number of fitted patient-specific parameters and modelling uncertainty was optimally balanced for the small patient cohort of 16 patients. This also limited the power of our analysis leading to no statistical significance of a potential benefit of iRT vs. HFSRT. Given the observed separation of the Kaplan-Meir plots for seven to 13 treatment fractions, our results are promising. There are a number of potential benefits of iRT over HFSRT which we will discuss in the following together with the relevant assumptions made upon modeling: i) possibility for dose escalation, ii) treatment personalization in reaction to the observed response, iii) repeated immune stimulation and antigen sampling, iv) maintenance of radiosensitive tumor subpopulation, v) tumor management rather than tumor eradication in case of a purely palliative treatment.

The clinical trial providing the data for this analysis demonstrated that it was safe to deliver 5×˜6 Gy as HFSRT with respect to normal tissue tolerance in recurrent HGG patients [8]. Addition of bevacizumab may have played a role in decreasing incidence of cerebral edema and radiation necrosis. Normal tissue toxicity following iRT+boost would need to be investigated for treatments comprising more than five fractions. Estimates on normal tissue complication could be related to previous results from trials investigating hypofractionated radiotherapy or stereotactic radiosurgery in combination with immunotherapy for the treatment of melanoma brain metastases [27, 28]. Severe radiation-induced late side effects of brain tissue may be beyond the expected life span of recurrent high-grade glioma patients, however, acute radiation-induced side effects such as headache, seizures, intracranial hemorrhage, and brain edema [27] should be considered. Besides these manageable toxicities, radiation necrosis may be a dose limiting factor for iRT treatments with incidence times in the order of months to few years following RT [28, 29]. Acute radiation-induced toxicity may strongly correlate with the irradiated volume and dosing which together with potential normal tissue recovery between fractions makes estimations difficult. A clear advantage of the intermittent treatment approach is the option to halt further irradiation if severe acute radiation-induced toxicity occurs, which is in line with a personalized treatment approach.

Another advantage of iRT is the possibility to adapt the PTV according to the observed growth. This includes local PTV adaptations, and potential inclusion of progression sites appearing outside the primary tumor location. This type of treatment paradigm would increase treatment cost due to repeated imaging and treatment planning. Recent advances in automated treatment planning [30, 31] and the delivery of the treatment under MRI guidance with an MR-Linac [32-34] could pose a potential solution to mitigate this limitation of intermittent treatments. Response monitoring and treatment planning steps could be combined in this scenario [35].

Additionally, intermittent RT may hold potential for synergistic action with immunotherapy due to repeated antigen re-sampling as suggested by recent (pre-) clinical studies [14-16, 50, 51]. Therefore, iRT is particularly promising in combination with immune-checkpoint inhibition therapy. In this approach bevacizumab and pembrolizumab treatments were modeled as additive effects to RT only. Moreover, whereas others explicitly modeled the drug administration schedule [52-54] we used a simplified model of bevacizumab and pembrolizumab administration, ignoring (patient-) specific pharmacodynamics as previously suggested [12] to limit the complexity of our model. Since in the NCT02313272 trial RT was only delivered in combination with bevacizumab and pembrolizumab it was not possible to separate a potential radiation-induced immune stimulation from direct radiation cytotoxicity. Since no further immune stimulation was modeled in the intermittent approach, it is suggested that the volume estimates made by our model may overestimate tumor growth dynamics and provide a worst-case scenario. While it is mathematically straightforward to describe immunostimulatory effects of radiation, the calibration and validation of the associated parameters proves infeasible and limits model predictive power [55].

It is important to clearly state the underlying assumptions made in our model which were required to limit the model's complexity and base the simulation only on the data available: i) use of an exponential tumor growth model, ii) tumor heterogeneity was ignored, iii) bevacizumab and pembrolizumab treatments were modelled as additive effects to RT only. By maintaining rather than eradicating the tumor, growth is likely to be slower for larger tumors than those close to eradication as presented in the NCT02313272 trial. As such, modelling exponential tumor growth for both, the HFSRT and iRT scenarios would in the worst case overestimate the growth of larger tumors present following iRT. It should also be stressed that the time between fractionations for iRT (six weeks) would allow for regrowth of both, resistant and sensitive populations. Pre-clinical data and evolutionary convention [36, 37] suggest that resistant cells may display a fitness disadvantage relative to sensitive clones in the absence of the selective pressure, allowing for the sensitive subpopulation to preferentially repopulate the tumor. Treatment prolongation by delivery of additional fractions would increase tumor radio resistance. However, it is expected that intermittent treatments, again, would provide an advantage over daily HFSRT as sensitive subclones may repopulate more effectively between fractions. This advantage would be more pronounced with increasing time between RT fractions. Pérez-García et al. specifically evaluated the impact of the treatment interval and concluded that the optimal timing depends on the individual tumor growth rate [9, 11]. In our study, a constant growth rate was used for all patients due to insufficient data to estimate tumor growth in the absence of treatment at an individual level. If the pre-treatment tumor growth rate was known, personalized adaptations of the fractionation interval would be possible allowing for longer intervalsin case of slow growing tumors. It should also be noted that RT-induced repeated antigen sampling may provide a specific immune stimulus targeting radio-resistant tumor subpopulations. This would be a further motivation for the combination of iRT with immune checkpoint inhibitors.

Finally, by maintaining rather than eradicating the tumor, growth is likely to be slower for larger tumors than those close to eradication as presented in the NCT02313272 trial. As such, modeling exponential tumor growth, one of the key assumptions within our model, for both the HFSRT and iRT scenarios would in the worst case overestimate the growth of larger tumors present following iRT. In summary, the assumptions in this analysis lead to a worst case estimate of the simulated treatment response following iRT, which strengthens the results presented in this hypothesis-generating study.

Patients with recurrent HGG currently have no curative treatment options [40, 41], and radical treatment attempting tumor control may be suboptimal considering its purely palliative and life-prolonging intent. The intermittent treatment approach embraces the aim of tumor control and volume management rather than tumor eradication. This comes at the cost of potentially not improving the tumor burden, which may affect the quality of life of the patients. For this reason, iRT should be restricted to those patients with asymptomatic recurrence or those who are neurologically stable. Our hypothesis generating modelling study provides a numerical estimate of the potential gain in time to progression for a large subgroup of patients. As such, we have demonstrated the mathematical feasibility of iRT treatments for recurrent HGG patients. This approach should be carried forward to be evaluated in a prospective clinical trial.

### Conclusion

There is a critical unmet clinical need to improve response rates and overall outcome for patients with recurrent HGG. Based on a deliberately simple, worst-case estimate mathematical model we propose that intermittent radiotherapy treatments with an optional three fraction boost may be a safe (in terms of tumor control) and potentially life prolonging treatment option for this group of patients. This novel radiation treatment schedule has additional potential for personalized treatment decisions in terms of geometric dose delivery and fraction size optimization based on the observed tumor response to previous fractions.

### REFERENCES

1. van Linde, M. E. et al. Treatment outcome of patients with recurrent glioblastoma multiforme: a retrospective multicenter analysis. J. Neuro-Oncology 135, 183-192, DOI: 10.1007/511060-017-2564-z (2017).

2. Chaul-Barbosa, C. & Marques, D. F. How We Treat Recurrent Glioblastoma Today and Current Evidence. Curr. Oncol. Reports 21, 94, DOI: 10.1007/511912-019-0834-y (2019).

3. Alexander, B. M. & Cloughesy, T. F. Adult Glioblastoma. J. clinical oncology: official journal Am. Soc. Clin. Oncol. 35, 2402-2409, DOI: 10.1200/JCO.2017.73.0119 (2017).

4. Network, N. C. C. Anaplastic gliomas/glioblastoma.

5. McGranahan, T., Therkelsen, K. E., Ahmad, S. & Nagpal, S. Current State of Immunotherapy for Treatment of Glioblastoma. Curr. Treat. Options Oncol. 20, 24, DOI: 10.1007/s11864-019-0619-4 (2019).

6. Laub, C. K., Stefanik, J. & Doherty, L. Approved Treatments for Patients with Recurrent High-grade Gliomas. Semin. Oncol. Nurs. 34, 486-493, DOI: 10.1016/j.soncn.2018.10.005 (2018).

7. Bahadur, S., Sahu, A. K., Baghel, P. & Saha, S. Current promising treatment strategy for glioblastoma multiform: A review. Oncol. Rev. 13, DOI: 10.4081/onco1.2019.417 (2019).

8. Sahebjam, S. et al. Hypofractionated Stereotactic Re-Irradiation with Pembrolizumab and Bevacizumab in Patients with Recurrent High Grade Gliomas: Results from a Phase 1 Study. Neuro-Oncology DOI: 10.1093/neuonc/noaa260 (2020).

9. Minniti, G. et al. Fractionated stereotactic reirradiation and concurrent temozolomide in patients with recurrent glioblastoma. J. Neuro-Oncology 103, 683-691, DOI: 10.1007/s11060-010-0446-8 (2011).

10. Connell, J. H. The Influence of Interspecific Competition and Other Factors on the Distribution of the Barnacle Chthamalus Stellatus. Ecology 42, 710-723, DOI: 10.2307/1933500 (1961).

11. Zeilinger, A. R., Olson, D. M. & Andow, D. A. Competitive release and outbreaks of non-target pests associated with transgenic Bt cotton. Ecol. Appl. 26, 1047-1054, DOI: 10.1890/15-1314 (2016).

12. Wargo, A. R., Huijben, S., de Roode, J. C., Shepherd, J. & Read, A. F. Competitive release and facilitation of drug-resistant parasites after therapeutic chemotherapy in a rodent malaria model. Proc. Natl. Acad. Sci. 104, 19914-19919, DOI: 10.1073/pnas.0707766104 (2007).

13. Zhang, J., Cunningham, J. J., Brown, J. S. & Gatenby, R. A. Integrating evolutionary dynamics into treatment of metastatic castrate-resistant prostate cancer. Nat. Commun. 8, 1816, DOI: 10.1038/s41467-017-01968-5 (2017).

14. Brady-Nicholls, R. et al. Prostate-specific antigen dynamics predict individual responses to intermittent androgen deprivation. Nat. Commun. 11, 1750, DOI: 10.1038/s41467-020-15424-4 (2020).

15. Niemierko, A. Reporting and analyzing dose distributions: A concept of equivalent uniform dose. Med. Phys. 24, 103-110, DOI: 10.1118/1.598063 (1997).

16. Niemierko, A. A Generalized Concept of Equivalent Uniform Dose (EUD) (1999).

17. Kim, M., Kotas, J., Rockhill, J. & Phillips, M. A Feasibility Study of Personalized Prescription Schemes for Glioblastoma Patients Using a Proliferation and Invasion Glioma Model. Cancers 9, 51, DOI: 10.3390/cancers9050051 (2017).

18. Wen, P. Y. et al. Updated Response Assessment Criteria for High-Grade Gliomas: Response Assessment in Neuro-Oncology Working Group. J. Clin. Oncol. 28, 1963-1972, DOI: 10.1200/JCO.2009.26.3541 (2010).

19. Glazar, D. J. et al. Tumor Volume Dynamics as an Early Biomarker for Patient-Specific Evolution of Resistance and Progression in Recurrent High-Grade Glioma. J. Clin. Medicine 9, 2019, DOI: 10.3390/jcm9072019 (2020).

20. Prokopiou, S. et al. A proliferation saturation index to predict radiation response and personalize radiotherapy fractionation. Radiat. Oncol. 10, 159, DOI: 10.1186/s13014-015-0465-x (2015).

21. Fowler, J. F. The linear-quadratic formula and progress in fractionated radiotherapy. The Br. J. Radiol. 62, 679-694, DOI: 10.1259/0007-1285-62-740-679 (1989).

22. Eriksson, D. & Stigbrand, T. Radiation-induced cell death mechanisms. Tumor Biol. 31, 363-372, DOI: 10.1007/s13277-010-0042-8 (2010).

23. Hubenak, J. R., Zhang, Q., Branch, C. D. & Kronowitz, S. J. Mechanisms of Injury to Normal Tissue after Radiotherapy. Plast. Reconstr. Surg. 133, 49e-56e, DOI: 10.1097/01.prs.0000440818.23647.0b (2014).

24. Creed, J., Gerke, T. & Berglund, A. MatSurv: Survival analysis and visualization in MATLAB. J. Open Source Softw. 5, 1830, DOI: 10.21105/joss.01830 (2020).

25. Eschrich, S. et al. Systems Biology Modeling of the Radiation Sensitivity Network: A Biomarker Discovery Platform. Int. J. Radiat. Oncol. 75, 497-505, DOI: 10.1016/j.ijrobp.2009.05.056 (2009).

26. Eschrich, S. A. et al. A Gene Expression Model of Intrinsic Tumor Radio sensitivity: Prediction of Response and Prognosis After Chemoradiation. Int. J. Radiat. Oncol. 75, 489-496, DOI: 10.1016/j.ijrobp.2009.06.014 (2009).

27. Diao, K. et al. Stereotactic radiosurgery and ipilimumab for patients with melanoma brain metastases: clinical outcomes and toxicity. J. Neuro-Oncology 139, 421-429, DOI: 10.1007/511060-018-2880-y (2018).

28. Prabhu, R. S. et al. Preoperative stereotactic radiosurgery before planned resection of brain metastases: updated analysis of efficacy and toxicity of a novel treatment paradigm. J. Neurosurg. 131, 1387-1394, DOI: 10.3171/2018.7.JNS181293 (2019).

29. Lawrence, Y. R. et al. Radiation Dose-Volume Effects in the Brain. Int. J. Radiat. Oncol. 76, S20—S27, DOI: 10.1016/j.ijrobp.2009.02.091 (2010).

30. Fan, J. et al. Automatic treatment planning based on three-dimensional dose distribution predicted from deep learning technique. Med. Phys. 46, 370-381, DOI: 10.1002/mp.13271 (2019).

31. Tsui, G. et al. Automated Machine-Learning Radiation Therapy Treatment Planning for Pediatric and Adult Brain Tumors. Int. J. Radiat. Oncol. 108, e777, DOI: 10.1016/j.ijrobp.2020.07.232 (2020).

32. Mutic, S. & Dempsey, J. F. The ViewRay System: Magnetic Resonance-Guided and Controlled Radiotherapy. Semin. Radiat. Oncol. 24, 196-199, DOI: 10.1016/j.semradonc.2014.02.008 (2014).

33. Lagendijk, J. J., Raaymakers, B. W. & van Vulpen, M. The Magnetic Resonance Imaging-Linac System. Semin. Radiat. Oncol. 24, 207-209, DOI: 10.1016/j.semradonc.2014.02.009 (2014).

34. Keall, P. J., Barton, M. & Crozier, S. The Australian Magnetic Resonance Imaging—Linac Program. Semin. Radiat. Oncol. 24, 203-206, DOI: 10.1016/j.semradonc.2014.02.015 (2014).

35. Enderling, H., Lopez Alfonso, J. C., Moros, E., Caudell, J. J. & Harrison, L. B. Integrating Mathematical Modeling into the Roadmap for Personalized Adaptive Radiation Therapy. Trends Cancer 5, 467-474, DOI: 10.1038/s41467-020-15424-4 (2019).

36. Gatenby, R. & Brown, J. The Evolution and Ecology of Resistance in Cancer Therapy. Cold Spring Harb. Perspectives Medicine 8, a033415, DOI: 10.1101/cshperspect.a033415 (2018).

37. Greaves, M. & Maley, C. C. Clonal evolution in cancer. Nature 481, 306-313, DOI: 10.1038/nature10762 (2012).

38. Sahebjam, S., Sharabi, A., Lim, M., Kesarwani, P. & Chinnaiyan, P. Immunotherapy and radiation in glioblastoma. J. Neuro-Oncology 134, 531-539, DOI: 10.1007/s11060-017-2413-0 (2017).

39. Marciscano, A. E. et al. Immunomodulatory Effects of Stereotactic Body Radiation Therapy: Preclinical Insights and Clinical Opportunities. Int. J. Radiat. Oncol. DOI: 10.1016/j.ijrobp.2019.02.046 (2019).

40. Seystahl, K., Wick, W. & Weller, M. Therapeutic options in recurrent glioblastoma—An update. Critical Rev. Oncol. 99, 389-408, DOI: 10.1016/j.critrevonc.2016.01.018 (2016).

41. Birk, H. S., Han, S. J. & Butowski, N. A. Treatment options for recurrent high-grade gliomas. CNS Oncol. 6, 61-70, DOI: 10.2217/cns-2016-0013 (2017).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method for assessing patient-specific evolution of resistance to therapy and progression of disease in recurrent high-grade glioma patients, comprising:
receiving a plurality of patient-specific parameters for a patient having recurrent high-grade glioma, wherein the patient-specific parameters comprise an evolution of resistance rate to a combination therapy, a pre-treatment tumor volume, and a radiation surviving fraction;
simulating, for each of a plurality of radiation therapy protocols, a respective volumetric tumor growth trajectory for the patient, wherein the simulation is performed using a tumor growth model based on the patient-specific parameters; and
determining an optimal radiation therapy protocol based on the simulation, wherein the optimal radiation therapy prolongs progression of the recurrent high-grade glioma.

2. The computer-implemented method of claim 1, wherein the plurality of radiation therapy protocols comprise hypofractionated stereotactic radiotherapy (HFSRT) and intermittent high dose radiotherapy (iRT).

3. The computer-implemented method of claim 2, wherein iRT is a high dose per fraction administered on a plurality of non-consecutive days.

4. The computer-implemented method of claim 3, wherein a time interval between radiation therapy treatments is between about 30 and 60 days.

5. The computer-implemented method of claim 2, wherein the optimal radiation therapy protocol is iRT, and wherein determining the optimal radiation therapy protocol comprises determining at least one of a dose per fraction, a number of fractions, or a time interval between radiation therapy treatment.

6. The computer-implemented method of claim 1, wherein the simulation is performed using the tumor growth model based on the patient-specific parameters and at least one of a tumor growth rate in an absence of therapy or an initial sensitivity to the combination therapy.

7. The computer-implemented method of claim 6, wherein at least one of the tumor growth rate in the absence of therapy or the initial sensitivity to the combination therapy is patient-specific.

8. The computer-implemented method of claim 1, wherein the combination therapy comprises immunotherapy and anti-angiogenic therapy.

9. The computer-implemented method claim 1, wherein the recurrent high-grade glioma is glioblastoma.

10. A method for treating a patient with recurrent high-grade glioma, comprising:
determining an optimal radiation therapy protocol according to claim 1; and
administering the optimal radiation therapy to the patient.

11. The method of claim 10, wherein the optimal radiation therapy is intermittent high dose radiotherapy (iRT).

12. The method of claim 10, further comprising administering a combination therapy to the patient in conjunction with iRT.

13. The method of claim 12, wherein the combination therapy comprises immunotherapy and anti-angiogenic therapy.

14. The method of claim 10, wherein the recurrent high-grade glioma is glioblastoma.

15. A system for assessing patient-specific evolution of resistance to therapy and progression of disease in recurrent high-grade glioma patients, comprising:
a processor; and
a memory operably coupled to the processor, the memory having computer-executable instructions stored thereon that, when executed by the processor, cause the processor to:
receive a plurality of patient-specific parameters for a patient having recurrent high-grade glioma, wherein the patient-specific parameters comprise an evolution of resistance rate to a combination therapy, a pre-treatment tumor volume, and a radiation surviving fraction;
simulate, for each of a plurality of radiation therapy protocols, a respective volumetric tumor growth trajectory for the patient, wherein the simulation is performed using a tumor growth model based on the patient-specific parameters; and
determine an optimal radiation therapy protocol based on the simulation, wherein the optimal radiation therapy prolongs progression of the recurrent high-grade glioma.

16. The system of claim 15, wherein the plurality of radiation therapy protocols comprise hypofractionated stereotactic radiotherapy (HFSRT) and intermittent high dose radiotherapy (iRT).

17. The system of claim 16, wherein the optimal radiation therapy protocol is iRT, and wherein determining the optimal radiation therapy protocol comprises determining at least one of a dose per fraction, a number of fractions, or a time interval between radiation therapy treatment.

18. The system of claim 15, wherein the simulation is performed using the tumor growth model based on the patient-specific parameters and at least one of a tumor growth rate in an absence of therapy or an initial sensitivity to the combination therapy.

19. The system method of claim 18, wherein at least one of the tumor growth rate in the absence of therapy or the initial sensitivity to the combination therapy is patient-specific.

20. The system of claim 15, wherein the recurrent high-grade glioma is glioblastoma.
